(19) United States
(12) Patent Application Publication
(10) Pub. No.: US 2021/0087620 A1
(43) Pub. Date: Mar. 25, 2021

(54) Target Gene Identifying Method for Tumor Treatment

(71) Applicant: Samsung Life Public Welfare Foundation Samsung Life Public Welfare Foundation, Seoul (KR)

(72) Inventors: Do-Hyun Nam, Seoul (KR); Jin Ku Lee, Seoul (KR); Jason Kyung Ha Sa, Seoul (KR)

(73) Assignee: Samsung Life Public Welfare Foundation, Seoul (KR)

(21) Appl. No.: 16/484,546

(22) PCT Filed: Feb. 5, 2018
(86) PCT No.: PCT/KR2018/001501

§ 371 Date: Aug. 8, 2019

(30) Foreign Application Priority Data
Feb. 9, 2017 (KR) 10-2017-0017998

(51) Int. Cl. C12Q 1/6869; C12Q 1/6886

(52) U.S. Cl. CPC C12Q 1/6869; C12Q 1/6886; C12Q 2600/136; C12Q 2600/106; C12Q 2535/101

(57) ABSTRACT

A target gene identifying method for tumor treatment according to the present invention comprises the steps of: taking multiple samples from a patent's tumor; analyzing the multiple samples for genetic variation: subjecting the multiple samples to drug screening to measure drug sensitivity of each sample; analyzing tumor heterogeneity on the basis of the genetic variation analysis result and the drug sensitivity measurement result; and identifying a target gene of the tumor on the basis of the tumor heterogeneity analysis result.

### TECHNICAL FIELD

The present disclosure relates to a method of identifying a target gene for tumor therapy, and more specifically, a method of identifying a target gene by collecting multiple tumor samples, and then identifying an ancestral mutation of a tumor through genetic variation analysis and drug screening.

### BACKGROUND ART

A tumor is a cell mass that grows abnormally due to a genetic alteration in cells. In this regard, starting from an ancestral genetic alteration that causes early tumorigenesis, various secondary genetic alterations occur, and a tumor may have various genetic alterations depending on the cells. For this reason, it is difficult to determine which genes should be targeted for treatment of such tumors.

For example, when a primary tumor and a secondary tumor develop in a patient, and the drug used to treat the primary tumor targets only the genetic alteration occurring in the primary tumor, this drug may exhibit no effect on the secondary tumor, and rather, may even cause the secondary tumor to develop. Therefore, it is important to know which genetic alterations are ancestral driver alterations.

Recently, methods of analyzing intratumor heterogeneity, which indicates diversity of tumor cells, have emerged. For example, Prior Document by Marco Gerlinger et al. discloses a method of analyzing phylogenetic relationships of tumors by extracting cells from multiple tumor sites, obtaining genetic information thereof, and analyzing private mutations of single cells among ubiquitous mutations common to every cell.

Similarly, US Patent Publication No. 2015-0227687 also discloses a system and a method for identifying intratumor heterogeneity using genetic information.

### DESCRIPTION OF EMBODIMENTS

### Technical Problem

However, the above methods are only for analyzing intratumor heterogeneity or phylogenetic relationships of tumors, and thus they do not suggest a method of identifying target genes for actually obtaining optimal therapeutic effects. Further, since genetic variation analysis has some inaccuracies, it is not always possible to fully analyze intratumor heterogeneity. In other words, there is a problem in that the existing method has no other way to verify whether the genetic variation analysis is correct.

To solve many problems including the above problem, an object of the present disclosure is to provide a method of identifying a target gene, in which an optimal therapeutic method may be suggested by identifying the target gene for complementary treatment of tumors through genetic variation analysis and drug screening. However, this object is merely illustrative, and the scope of the present disclosure is not limited thereto.

### Solution to Problem

A method of identifying a target gene for tumor therapy according to the present disclosure may include collecting multiple samples from a patients tumor; analyzing genetic variations of the multiple samples; measuring drug sensitivity of each sample by subjecting the multiple samples to drug screening; analyzing intratumor heterogeneity of the tumor on the basis of the result of analyzing the genetic variations and the result of measuring drug sensitivity; and identifying the target gene of the tumor on the basis of the result of analyzing the intratumor heterogeneity.

The collecting of the multiple samples may be collecting of samples from different sites of the patient' tumor.

The collecting of the multiple samples may be collecting of each sample from the patient' tumors developing at different times.

The analyzing of genetic variations of the multiple samples may be performed by massive sequencing analysis (next-generation sequencing, NGS).

A drug used in the measuring of drug sensitivity may be an anticancer agent.

The measuring of drug sensitivity of each sample by subjecting the multiple samples to drug screening may include obtaining a cell viability curve of each sample according to a dose of each drug; and calculating an area under the curve.

The identifying of the target gene of the tumor may include measuring a variance and a mean value of the drug sensitivity for each sample; and selecting a drug with the highest mean value of the drug sensitivity, among drugs having a variance lower than a predetermined value.

The analyzing of intratumor heterogeneity may include analyzing intratumor heterogeneity on the basis of the result of analyzing the genetic variations; and verifying the intratumor heterogeneity on the basis of the result of measuring the drug sensitivity.

Aspects, features, and advantages other than those described above will become apparent from the following drawings, claims, and detailed description.

### Advantageous Effects of Disclosure

According to the present disclosure, genetic variation analysis and drug sensitivity measurement through drug screening may be performed in a complementary manner for multiple samples, thereby identifying ancestral driver mutation with higher accuracy than existing methods. Therefore, it is possible to provide a method of identifying a target gene for tumor therapy with higher reliability. However, the scope of the present disclosure is not limited by these effects.

### BRIEF DESCRIPTION OF DRAWINGS

FIG. 1 is a flowchart showing a schematic illustration of a method of identifying a target gene for tumor therapy according to the present disclosure;

FIG. 2 shows different methods of collecting multiple samples;

FIG. 3 is an experimental example showing results of analyzing genetic variations of a GBM9 patient according to single cell analysis: and FIG. 4 is a topological graph depicting intratumor heterogeneity of the GBM9 patient on the basis of the above results;

FIG. 5 is an experimental example showing results of analyzing genetic variations of the GBM19 patient according to bulk tumor tissue and cell analysis;

FIG. 6 is an experimental graph showing survival rates of sample tumor cells according to doses of three kinds of drugs for the GBM9 patient; FIG. 7 shows an experimental graph showing drug sensitivity for the left and right tumor cells according to doses of 40 kinds of drugs for the GBM9 patient; and

FIG. 8 shows tumor phylogeny on the basis of the result of analyzing intratumor heterogeneity of the GBM9 patient.

### BEST MODE

The present disclosure may be variously modified and may have various embodiments, and thus specific embodiments will be illustrated in drawings and explained in a detailed description. Effects and features of the present disclosure and methods of accomplishing the same may be understood more readily by reference to the following detailed description of embodiments and the accompanying drawings. The present disclosure may, however, be embodied in many different forms and should not be construed as being limited to the embodiments set forth below.

The term ‘mutation’ or ‘variation’ refers to a state in which DNA on which genetic information is recorded has changed from the original due to various factors, and may include all kinds of mutations occurring at a nucleotide level such as point mutation, insertion, deletion, etc. as well as mutations occurring at a chromosome level such as gene duplication, gene deletion, chromosomal inversion, etc.

In the following embodiments, the term “first”, “second”, or the like is employed not for purposes of limitation, but to distinguish one element from another.

In the following embodiments, the singular expression may include the plural expression unless it is differently expressed contextually.

In the following embodiments, the term such as “including”, “having”, etc. includes the presence of features or components described herein, but not to preclude the addition of one or more other features or components.

Hereinafter, embodiments of the present disclosure will be described in detail with reference to the accompanying drawings. In describing with reference to the drawings, the same or corresponding elements are given the same reference numerals, and a repetitive description thereof will be omitted.

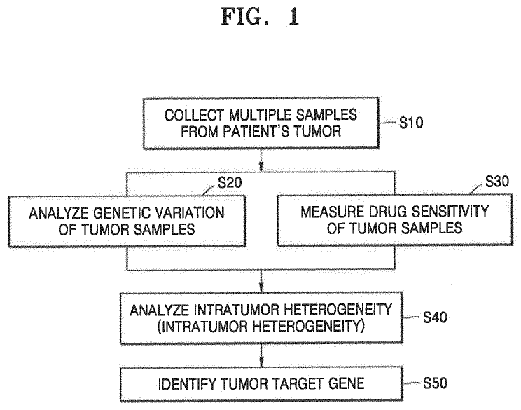

FIG. 1 is a flowchart showing a schematic illustration of a method of identifying a target gene for tumor therapy according to the present disclosure.

The method of identifying a target gene for tumor therapy according to the present disclosure may include collecting multiple samples from a patients tumor (S10); analyzing genetic variations of the multiple samples (S20); measuring drug sensitivity of each sample by subjecting the multiple samples to drug screening (S30); analyzing intratumor heterogeneity on the basis of the result of analyzing the genetic variations and the result of measuring drug sensitivity (S40); and identifying the target gene of the tumor on the basis of the result of analyzing the intratumor heterogeneity (S50).

Referring to FIG. 1, the collecting of multiple samples from the patient's tumor (S10) may be performed. In the present disclosure, the tumor refers to a cell mass that abnormally grows due to genetic alteration of cells.

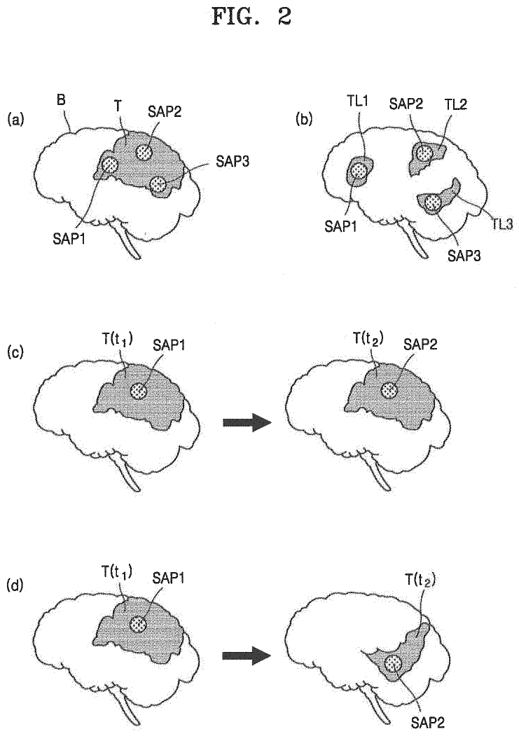

FIG. 2 shows different methods of collecting multiple samples.

According to one embodiment, the collecting of the multiple samples may be collecting of samples from different sites of the patient' tumor.

Referring to (a) of FIG. 2, for example, when a tumor (T) develops at a particular region of the brain (B), samples of tumor (T) may be collected from multiple sample acquisition points (SAPs). In (a) of FIG. 2, for example, respective samples may be collected from three sample acquisition points (SAP1, SAP2, and SAP3).

Referring to (b) of FIG. 2, when several tumor lesions (TLs) develop in the brain, tumor samples may be collected from each of the tumor lesions. In (b) of FIG. 2, for example, when three tumor lesions (TL1, TL2, and TL3) develop, respective samples may be collected from sample acquisition points (SAP1, SAP2, and SAP3) of each lesion.

In other words, as in (a) and (b) of FIG. 2, it is possible to collect several samples from spatially different sites.

According to one embodiment, the collecting of the multiple samples may be collecting of the respective samples from the patient' tumors which develop at different times. In other words, it is possible to collect several samples at temporally different times. For example, there is a case that after tumorectomy of a primary tumor, recurrent tumor may occur over time. In this regard, a tumor T(t₁) occurring at a first time (t₁) and a tumor T(t₂) occurring at a second time (t₂) may occur at the same site as shown in (c) of FIG. 2 or may occur at different sites as shown in (d) of FIG. 2. In either case, respective samples may be collected from sample acquisition points (SAP1 and SAP2) of each of tumor T(t₁) and tumor T(t₂).

These methods of collecting samples as in (a), (b), (c), and (d) of FIG. 2 may be performed in combination. For example, FIG. 4 shows a tumor MRI image of glioblastoma patient No. 9 (GBM9) used in Experimental Example of the present disclosure. In this patient, each one tumor (GBM9-1 and GBM9-2) emerged in the right and left frontal lobes, and recurrent tumors (GBM9-R1 and GBM9-R2) emerged in the left frontal lobe after concurrent chemoradiotherapy (CCRT) and EGFR targeted treatment. At this time, samples were collected from tumors (GBM9-1, GBM9-2, GBM9-R1, and GBM9-R2) that occurred at spatially different sites and temporally different times, respectively, thereby obtaining multiple samples.

The reason for collecting multiple samples from tumors is to analyze the intratumor heterogeneity using both results of genetic variation analysis and drug sensitivity test, which will be described below.

Referring to FIG. 1, the analyzing of genetic variations of the multiple samples (S20) may be performed. The analyzing of genetic variations may include analyzing of base sequences of genes of the sample cells.

According to one embodiment, the analyzing of base sequences may be performed by, for example, massive sequencing analysis (next-generation sequencing, NGS). Meanwhile, the analyzing of base sequences may be performed by Whole exome sequencing (WES). Exome which is a protein-coding region occupies about 2% of the whole human genome, but about 85% of disease-related genes known until now are located on the exome. For sequencing of only the exome, it is necessary to isolate only the exome from the whole genome. Various methods such as a solution-based capture method of mixing a sample with a bait probe corresponding to the exome, an array-based capture method of extracting the exome by binding a probe to a chip, a PCR method, etc. may be employed. In addition, various techniques of analyzing sequences of DNA, RNA, or transcriptome may be used to analyze genetic variations of the tumor sample cells,

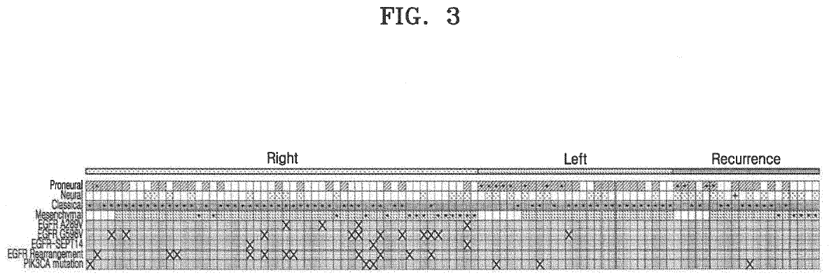

FIG. 3 is an experimental example showing the results of analyzing genetic variations of the GBM9 patient according to single cell analysis, and FIG. 4 is a topological graph depicting intratumor heterogeneity of the GBM9 patient on the basis of the above results.

Since cells divide every hour and every minute, even the same tumor cells may have different clones. In other words, although a tumor sample is collected from one patient, individual cells may have different genetic variations, which is called intratumor heterogeneity. In this regard, to analyze genetic variations of a number of cells, multiple samples are needed.

FIG. 3 shows expression profiles of individual tumor cells obtained from three samples which were extracted from right, left, and recurrent tumors of the GBM9 patient. For each cell, a subtype with the highest expression is marked with a dot (⋅). EGFR genomic alterations are marked with X.

By comparing similarity between expression data of individual cells, topological representation of each tumor cell may be obtained as in FIG. 4. In FIG. 4, each node represents clustering of cells having similar variation from the result of analyzing genetic variations, and a size of each node is proportional to the number of similar cells. A cell may appear in several nodes, and nodes are connected by a line if they have cells in common.

As shown in FIG. 4, cells extracted from each tumor of the GBM9 patient are clustered in the similar sites. Meanwhile, the left tumor and the recurrent tumor are overlapped with each other, implying that the recurrent tumor may arise from the left tumor of the BMS patient.

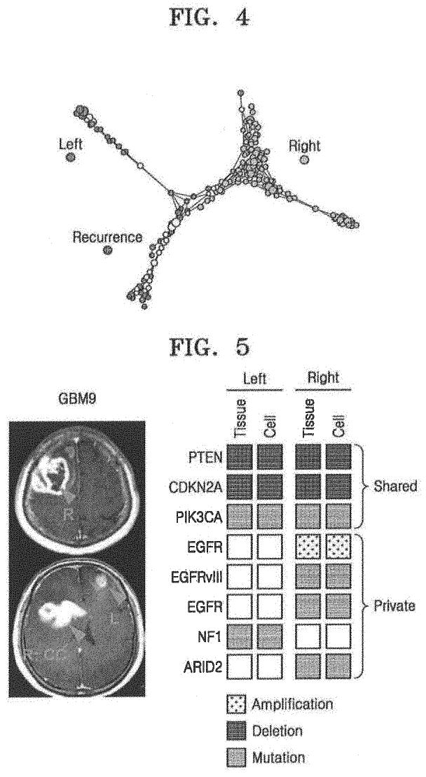

FIG. 5 is an experimental example showing the results of analyzing genetic variations of the GBM9 patient according to bulk tumor tissue and cell analysis. In bulk tumor tissue and cell analysis, when genetic variation occurs in a part of several cells, it suggests that the variation occurs in a particular cluster. The right figure of FIG. 5 shows results of analyzing genetic variations of tissues and cells of left and right tumors of the GBM9 patient. In the above patient, deletion of PTEN and CDKN2A genes and mutation of PIK3CA gene were found in all of the left and right tumors. Meanwhile, NF1 gene mutation was found only in the left tumor, and EGFR gene amplification, EGFRvIII gene mutation, EGFR gene mutation, and ARID2 gene mutation were found only in the right tumor.

As above, single cell analysis and/or bulk cell analysis may be used to analyze genetic variations of the sample tumors. However, it is not always possible to completely analyze intratumor heterogeneity by the above analysis methods. For example, referring to FIG. 3, when whether or not genetic variations occurred may not be determined by the single cell analysis, it is represented by gray color. That is, the intratumor heterogeneity graph of FIG. 4 which was analyzed based on the result involving a lot of missing data may have some errors.

Like this, when genetic variations of tumors are analyzed by bulk tumor tissue and cell analysis, some errors may also be caused. For example, when data show that a mutation rate of a specific gene in some cells of a tumor is low, it may be difficult to determine whether this is actually a mutation or an error in a measuring device. Therefore, another method of verify whether or not the gene mutation actually occurred in the tumor is also needed.

According to the present disclosure, separately from the analyzing of genetic variations (S20), the measuring of drug sensitivity of each sample by subjecting the multiple samples to drug screening (S30) may be performed. Both of (S20) and (S30) may be performed at the same time as in FIG. 2, or any one of them may be performed before the other.

The drug screening is a process of assessing pharmacological activity or toxicity of synthetic compounds or natural products that are drug candidates. In the present disclosure, a drug used in the drug screening may be an anticancer agent. For example, the drug may be an inhibitor for inhibiting tumor metabolism. <Table 1> below is a table representing kinds of the inhibitors and targets thereof.

TABLE 1

| No. | Compound name | Generic name | Target | Clinical Phase |
|---|---|---|---|---|
| 1 | ABT-199 | GDC-0199 | Bcl-2 | Phase 3 |
| 2 | BIBW2992 | Afatinib | EGER | FDA Approved |
| 3 | AG013736 | Axitinib | VEGFR1/2/3, PDGFRβ | FDA Approved |
| | | | and c-Kit | |
| 4 | AZD2014 | | mTOR | Phase 2 |
| 5 | AZD4547 | | FGFR1/2/3 | Phase 2/3 |
| 6 | AZD5363 | | Akt1/2/3 | Phase 2 |
| 7 | AZD6244 | Selumetinib | MEK1 | Phase 3 |
| 8 | BEZ235 | | PI3K/mTOR | Phase 2 |
| 9 | BGJ398 | | FGFR1/2/3 | Phase 2 |
| 10 | BKM120 | Buparlisib | PI3K | Phase 2 |
| 11 | BMS-599626 | | EGFR | Phase 1 |
| 12 | bosutinib | Bosutinib | dual Src/Abl | FDA Approved |
| 13 | BYL719 | | PI3K | Phase 2 |
| 14 | XL184 | Cabozantinib | VEGFR2, c-Met, Ret, Kit, | FDA Approved |
| | | | Flt-1/3/4, Tie2 | |
| 15 | AZD2171 | Cediranib | VEGFR, Flt | Phase 3 |
| 16 | CI-1033 | Canertinib | EGFR, HER2 | Phase 3 |
| 17 | CO-1686 | | EGFR | Phase 2 |
| 18 | PF-02341066 | Crizotinib | Met, ALK | FDA Approved |
| 19 | PF299804 | Dacomitinib | EGFR | Phase 2 |
| 20 | BMS-354825 | Dasatinib | Bcr-Abl | FDA Approved |
| 21 | TKI-258 | Dovitinib | Flt3, c-Kit, FGFR1/3, | Phase 4 |
| | | | VEGFR1/2/3, PDGFR | |
| 22 | | Erlotinib | HER1/EGFR | FDA Approved |
| 23 | RAD001 | Everolimus | mTOR | FDA Approved |
| 24 | XL880 | Foretinib | HGFR and VEGFR, | Phase 2 |
| | | | mostly for Met and KDR | |
| 25 | | Gefitinib | EGFR | FDA Approved |
| 26 | | Ibrutinib | Btk, Bmx, CSK, FGR, | FDA Approved |
| | | | BRK, HCK, less potent | |
| | | | to EGFR, ErbB2, JAK3 | |
| 27 | sti571 | Imatinib | v-Abl, c-Kit and PDGFR | FDA Approved |
| 28 | INCB28060 | Capmatinib | Met | Phase 1 |
| 29 | | Lapatinib | EGFR | FDA Approved |
| 30 | HKI-272 | Neratinib | EGFR | FDA Approved |
| 31 | AZD2281 | Olaparib | PARP1/2 | Phase 3 |
| 32 | | Pazopanib | VEGFR1/2/3, PDGFR, | FDA Approved |
| | | | FGFR, c-Kit | |
| 33 | PF-05212384 | | PI3K/mTOR | Phase 2 |
| | (PKI-587) | | | |
| 34 | | Ruxolitinib | JAK1/2 | FDA Approved |
| 35 | | Sunitinib | VEGFR2 and PDGFRβ | FDA Approved |
| 36 | MLN518 | Tandutinib | FLT3, PDGFR, and KIT | Phase 2 |
| 37 | AV-951 | Tivozanib | VEGFR, c-Kit, PDGFR | Phase 3 |
| 38 | | Trametinib | MEK1/2 | FDA Approved |
| 39 | ZD6474 | Vandetanib | VEGFR2 | FDA Approved |
| 40 | XL147 | | PI3K | Phase 1/2 |

However, the inhibitors used in the drug screening are not limited thereto.

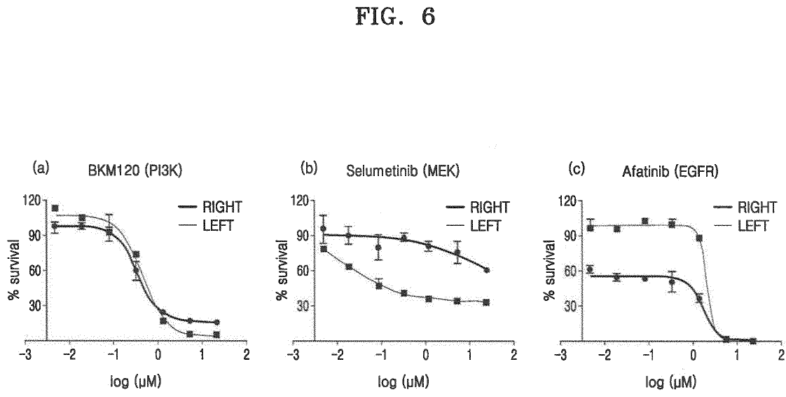

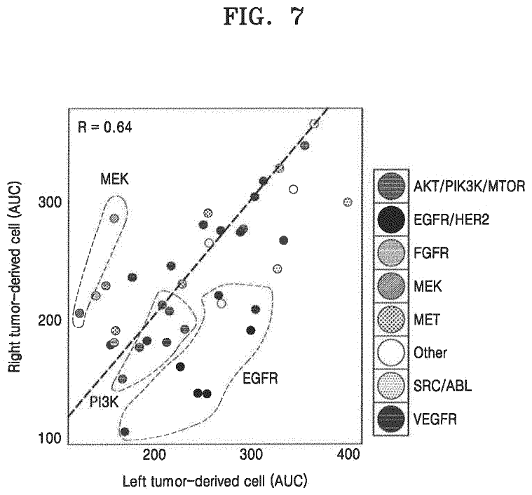

FIG. 6 is an experimental graph showing survival rates of sample tumor cells according to doses of three kinds of drugs for the GBM9 patient.

According to one embodiment, the measuring of drug sensitivity of each sample by subjecting the multiple samples to drug screening may include obtaining a cell viability curve of each sample according to a dose of each drug; and calculating an area under the curve.

GBM9 patient had tumors in the right and left frontal lobes of the brain, respectively. In this regard, 40 kinds of anticancer agents were administered to samples collected from the respective tumors, and tumor cell viability was examined. (See FIG. 7) The results of screening only three kinds of drugs (BKM120, Selumetinib, and Afatinib) are shown in FIG. 6.

When a curve of tumor cell viability vs drug dose is plotted, an area under the curve (AUC) may be used as an index of drug sensitivity. A low AUC value indicates that tumor cell viability decreased by the drug, indicating increased drug sensitivity.

Referring to (a) of FIG. 6, the graph shows survival rates of the left and right tumors of the GBM9 patient in response to BKM120 which is a drug inhibiting PI3K pathway of PIK3CA mutation. It was found that as the dose of BKM120 increased (X-axis direction), survival rates of the two tumors decreased. In other words, a relatively low area under the curve (AUC) values were obtained for both of the tumor samples, indicating high drug sensitivity of BKM120. Therefore, it implies that mutations associated with PIK3CA pathway occurred in both of the tumors.

Referring to (b) of FIG. 6, the graph shows survival rates of the left and right tumors of the GBM9 patient in response to selumetinib which is a drug inhibiting RAS/RAF/MEK/ERK pathway of NF1 mutation. Unlike (a) of FIG. 6, although the dose of selumetinib increased, the survival rate of the right tumor did not greatly decrease whereas the survival rate of the left tumor greatly decreased. In other words, the area under the curve (AUC) of the left tumor was lower than that of the right tumor, indicating high drug sensitivity of selumetinib for the left tumor. Therefore, it implies that NF1 mutation associated with RAS/RAF/MEK/ERK pathway occurred only in the left tumor.

Referring to (c) of FIG. 6, the graph shows survival rates of the left and right tumors of the GBM9 patient in response to afatinib which is a drug inhibiting EGFR overexpressed by EGFR mutation. Unlike (a) and (b) of FIG. 6, although the dose of afatinib is low, the survival rate of the right tumor maintained low until a predetermined dose (about 0 μM) whereas the survival rate of the left tumor maintained high. In other words, the area under the curve (AUC) of the right tumor was lower than that of the left tumor, indicating high drug sensitivity of afatinib for the right tumor. Therefore, it implies that the mutation associated with EGFR pathway occurred only in the right tumor.

FIG. 7 shows an experimental graph showing drug sensitivity for the left and right tumor cells according to doses of 40 kinds of drugs for the GBM9 patient. 40 kinds of the drugs (anticancer agents) are classified into 8 groups according to target genes (or inhibitors). The X-axis represents AUC values of the left tumor-derived cells for each drug, and the Y-axis represents AUC values of the right tumor-derived cells for each drug.

As an experimental result, data of the drugs that function as MEK inhibitors are mostly shown at the top left of the graph. In other words, the AUC values of the right tumors are high and the AUC values for the left tumors are low. This means that drug sensitivity for the right tumor is low, and drug sensitivity for the left tumor is high. The drugs that function as MEK inhibitors mainly act on the left tumors, indicating that NF1 gene mutation causing abnormality in RAS/RAF/MEK/ERK pathway occurred in the left tumors.

Meanwhile, data of the drugs that function as EGFR inhibitors are mostly shown at the bottom right of the graph. In other words, the AUC values of the left tumors are high and the AUC values for the right tumors are low. This means that drug sensitivity for the left tumor is low, and drug sensitivity for the right tumor is high. The drugs that function as EGFR inhibitors mainly act on the right tumors, indicating that EGFR gene mutation occurred in the right tumors.

Meanwhile, data of the drugs that inhibit PI3K pathway are mostly shown at the bottom left of the graph. In other words, all of the left and right tumors have similar AUC values. This means that drug sensitivity for the left and right tumors is similar. That is, PI3KCA gene mutation causing abnormality in PI3K pathway occurred in all the left and right tumors.

When multiple samples are sensitive to all the drugs used in the drug screening, it indicates that genetic mutations targeted by the drugs occurred in all the tumor sites from which the samples were collected.

These results of FIG. 7 are consistent with the results of analyzing genetic variation of FIG. 5. Both of the methods showed that PIK3CA mutation corresponds to ancestral mutation, and EGFR and MEK mutations occurred later, and thus each of the analysis results may be verified. In the absence of such a verification procedure, there is a possibility of misidentifying the target gene for tumor therapy. This will be described below.

According to one embodiment, the analyzing of intratumor heterogeneity (FIG. 1, S40) may include analyzing intratumor heterogeneity on the basis of the result of analyzing the genetic variations, and verifying the result of analyzing intratumor heterogeneity on the basis of the result of measuring drug sensitivity. In other words, intratumor heterogeneity may be analyzed on the basis of the result of analyzing the genetic variations of tumors through single cell analysis, bulk cell analysis, etc., and then this result may be verified on the basis of the result of measuring drug sensitivity.

Subsequently, referring to FIG. 1, the identifying of the target gene of the tumor on the basis of the result of analyzing the genetic variations and the result of measuring drug sensitivity (S50) may be performed. For example, on the basis of the results of FIGS. 5 and 7, it may be determined that PIK3CA gene is needed as a target for treating the GBM9 patient.

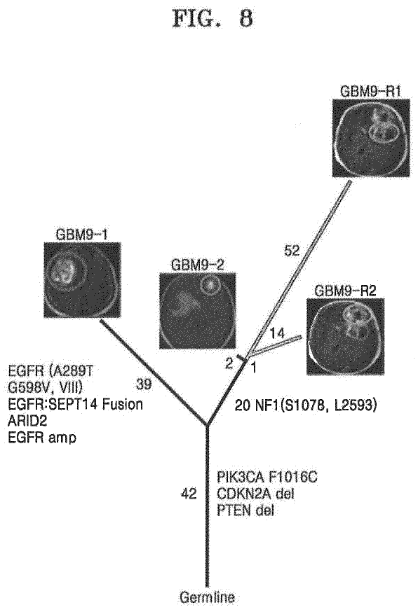

FIG. 8 shows tumor phylogeny on the basis of the result of analyzing intratumor heterogeneity of the GBM9 patient and the result of measuring drug sensitivity. For example, in the GBM9 patient, PTEN and CDKN2A deletion, and PIK3CA mutation occurred in the primary tumor, and then NF1 mutation occurred in the cells of the left tumor in a branch, and EGFR mutation occurred in the cells of the right tumor in another branch.

In this regard, for the treatment of both the left and right tumors in the GBM9 patient, a drug targeting PTEN gene deletion, CDKN2A gene deletion, or PIK3CA mutation corresponding to the ancestral mutation of the tumors, i.e., BKM120 is required to be administered.

However, before verifying the result of analyzing intratumor heterogeneity on the basis of drug sensitivity measurement, the GBM9 patient has been practically treated with afatinib. 1 month after treatment, the right tumor was treated, but afatinib targeting EGFR mutation did not exhibit efficacy on the left tumor having no EGFR mutation, and recurrent tumors occurred.

In other words, when ancestral mutation is identified by using both the genetic variation information and the results of measuring drug sensitivity, the target gene for tumor therapy may be accurately identified based on the ancestral mutation.

According to one embodiment, the identifying of the target gene of the tumor may include measuring a variance and a mean value of the drug sensitivity for each sample; and selecting a drug with the highest mean value of the drug sensitivity, among drugs having a variance lower than a predetermined value.

Referring to FIG. 7, in the case of the GBM9 patient, data near the dotted line show small variance of drug sensitivity or AUC, and data farther away from the dotted line show larger variance of drug sensitivity. The small variance means that the drug evenly acts on most of the samples. Therefore, to identify a target gene, it is necessary to select those having small variance of drug sensitivity. In this regard, the predetermined value may be appropriately selected depending on the kind of the drug, the kind of the tumor, etc.

Meanwhile, to select a drug that evenly acts on most of the samples, it is necessary to select those having a high mean value of drug sensitivity. For example, this means drugs locating near the dotted line and at the bottom left of the graph of FIG. 6. Meanwhile, the process of selecting such a drug may be performed through computation of a computer included in an analyzer.

According to the present disclosure, genetic variation analysis and drug sensitivity measurement through drug screening may be performed in a complementary manner for multiple samples, thereby identifying ancestral mutation with higher accuracy than existing methods. Therefore, it is possible to provide a method of identifying a target gene for tumor therapy with higher reliability.

The experimental results and graphs of the GBM9 patient are only for illustrating the present disclosure, and the scope of the present disclosure is not limited thereto.

### MODE OF DISCLOSURE

### Example

Acquisition and Culture of Glioma Specimens

The present inventors analyzed somatic variants in 127 tumor specimens from 52 glioma patients undergoing surgery at Samsung Medical Center (SMC). At this time, tumors were classified into 4 groups according to methods of collecting the samples (see FIG. 2). Samples of about 5×5×5 mm³ used for genomic analysis were snap-frozen using liquid nitrogen. Portions of the samples were enzymatically dissociated into single cells. The tumor cells were cultured in neurobasal media containing N2 and B27 supplements (0.5× each, Invitrogen) and human recombinant basic fibroblast growth factor (bFGF) and epidermal growth factor (EGF, 20 ng/ml each, R&D Systems). The patient-derived cells (PDCs) used here had shown no contamination of mycoplasma.

Whole Exome Sequencing

Raw Data

Agilent SureSelect kit was used for capturing exonic DNA fragments. Illumine HiSeq2000 was used for sequencing, and generated 2×101 bp paired-end reads.

Somatic Mutation

The sequenced reads in FASTQ files were aligned to the human genome assembly (hg19) using Burrows-Wheeler Aligner ver. 0.6.2. The initial alignment BAM files were subjected to preprocessing before mutation calling, such as sorting, removing duplicated reads, and locally realigning reads around potential small indels (insertion&deletion) (SAMtools, Picard ver. 1.73 and Genome Analysis Toolkit (GATK) ver. 2.5.2. were used)

The present inventors used MuTect (ver. 1.1.4) and Somatic IndelDetector (GATK ver. 2.2) to make high-confidence predictions on somatic mutations from the neoplastic and non-neoplastic tissue pairs. Variant Effect Predictor (VEP) ver. 73 was used to annotate the called somatic mutations. Additionally, Statistical Variant Identification (SAVI) software was run to call somatic variants and indels for refining the existing mutation calls.

Copy Number

An ngCGH python package and an excavator were used to generate estimated copy number alterations in tumor specimens as compared with its non-neoplastic part. The copy number of each gene was calculated by analyzing mean values of all exonic segments. When loge fold-change of tumor divided by normal is larger than 1, the gene was labeled as ‘amplified’, and when it was smaller than −1, the gene was labeled as ‘deleted’.

Cancer Cell Fractions and Clonality

The present inventors ran ABSOLUTE using input of genomic variants and copy number data to infer sample purity and cancer cell fractions (CCF) and removed those having purity of less than 20%.

They considered the corresponding mutations as clonal if 1) indicated “clonal” in ABSOLUTE program and with a cancer cell fraction of 80% or more or 2) having a cancer cell fraction of 100% and not marked as “clonal” or “subclonal”.

In the ABSOLUTE program, most gene mutations were indicated “subclonal” in hypermutated GBM18 initial and TCGA-14-1402 2^{nd} recurrence samples, and the reason is that the large mutational load may skew estimates. In hypermutated samples, treatment-associated mutation coupled with defects in mismatch repair are the most largely responsible. Therefore, mutations having CCF greater than or equal to the maximum mismatch repair CCF were marked ‘clonal’ in these two samples.

Nei Genetic Distances

Samples containing the spatial or longitudinal category were retained for statistical comparisons. Thereafter, Nei distance of CCF was calculated for each patient's sample as in the following <Equation 1>, wherein X=CCF of sample 1 and Y=CCF of sample 2.

$$D = - 1 * \frac{log ( X * Y + ( 1 - X ) * ( 1 - Y )}{\sqrt{\sum {X}^{2} + \sum 1 - {X}^{2} + \sum {Y}^{2} + \sum 1 - {Y}^{2}}} \qquad 〈 Equation 1 〉$$

RNA Sequencing

The trimmed sequence reads of 30 nucleotides (nt) were mapped on hg19 using GSNAP (ver. 2012-12-20), not allowing any mismatches, indels, or splicing. SAM files were aligned using SAMtools and summarized into BED files using bedTools (bamToBed. Ver. 2.16.2). R package DEGseq was used to estimate RPKM values. For analysis of gene fusion, reads crossing the fusion junction were separated, and fusion events were extracted using the same reference as in exon-skip analysis.

Isolation of Single Cells and RNA Sequencing

The present inventors used a C1TM Single-Cell Auto Prep System (Fluidigm) with a SMARTer kit (Clontech) to generate cDNAs from single cells. 352R and L cells were captured in C1 chip (17 μm to 25 μm) determined by microscopic examination as previously described. RNAs from samples were processed using the SMARTer kit with 10 ng of starting materials. Libraries were generated using a Nextera XT DNA Sample Prp Kit (Illumina) and sequenced on HiSeq 2500 using a 100 bp paired-end mode of TruSeq Rapid PECluster kit and Tru Seq Rapid SBS kit. Before mapping RNA sequencing reads to the reference, reads were filtered out at Q33 by using Trimmomatic-0.30. TPM values were calculated from each single cell using RSEM (ver. 1.2.25) and expressed as log₂ (1+TPM).

Gene Fusion Detection

Chimerascan was applied to generate candidate list of gene fusions. For bulk sequencing, only previously reported in-frame, high expressing fusions, such as FGFR3-TACC3, MGMT fusion, EGFR-SEPT14, and ATRX fusion were considered. For single cell fusion analysis, if a fusion was highly expressed and independently detected in other cells, the fusion will be reported.

Expression Based Subtypes Determination

Gene expression was measured by RSEM and then loge transformed. To determine the expression-based subtype of GBM cells, z-scores for gene expression data across samples were calculated, and then applied ssGSEA (ver. gsea2-2.2.1) on the normalized expression profile. For each cell, all genes were ranked based on their expression values to create a .rnk file as the input of the software GseaPreranked. An enrichment score was computed for all four subtypes defined in the prior document of Verhaak, R. G. et al. The subtype with the maximal enrichment score was used as the representative subtype for each cell.

Topological Data Analysis Using Single Cell Transcriptome

Normal cells were filtered out based on expression profile. To this end, expression signatures of normal oligodendrocytes, neurons, and astrocytes, microglia, endothelial cells, T-cells, and other immune cells were analyzed, and a Gaussian mixture model was used to classify individual cells according to their expression profile. 94/133, 82/85 and 90/137 cells, respectively for GBM9, GBM10, and GBM2, were classified as tumor cells.

After normalization of gene expression level by dividing total number of reads in each cell to eliminate the bias caused by batch effect, topological representations of these single cell data were built using Mapper algorithm, as implemented by Ayasdi Inc. Open-source of this algorithm is available from http://danifold.net/mapper, http://github.com/MLWave/kepler-mapper. The first two components of multidimensional scaling (MDS) were used as auxiliary functions for the algorithm. The output of Mapper is a low-dimensional network representations of the data. Nodes represent sets of cells with similar global transcriptional profiles (as measured by the correlation of the expression levels of the 2,000 genes with highest variance across each patient). Thereafter, individual genes that had an expression pattern localized in the network were identified and used to determine the sub-clonal structure of the samples at the level of expression.

PDC-Based Chemical Screening and Analysis

PDCs grown in serum-free medium were seeded in 384 well plates at a density of 500 cells per well in duplicate or triplicate. The drug panel consisted of 40 anticancer agents (Selleckchem) targeting oncogenic signals. Two hours after the plating. PDCs were treated with drugs in a four-fold and seven-point serial dilution from 20 μM to 4.88 nM using Janus Automated Workstation (PerkinElmer, Waltham, Mass., USA). After 6 days of incubation at 37° C. in a 5% CO₂ humidified incubator, cell viability was analyzed using an adenosine triphosphate (ATP) monitoring system based on firefly luciferase (ATPLite™ 1step, PerkinElmer). At this time, viable cells were estimated using an EnVision Multilabel Reader (PerkinElmer). Dimethyl sulfoxide (DMSO) was also included as control in each plate. Controls were used for calculation of relative cell viability for each plate and plate normalization. DRC fitting was performed using GraphPad Prism 5 (GraphPad) and evaluated by measuring an area under the curve (AUC) of dose response curve. After normalization, best-fit lines were determined and the AUC value of each curve was calculated using a GraphPad Prism. At this time, regions defined by fewer than two peaks were ignored. Cell viability was determined by calculating AUC values of dose-response curves (DRCs) with exclusion of non-convergent fits.

Although the present disclosure has been described with reference to embodiments shown in the drawings, these are only illustrative, and those skilled in the art will appreciate that various changes and equivalents thereto may be made. Therefore, the technical scope of protection of the present disclosure is defined by the technical scope of the appended claims.

### INDUSTRIAL APPLICABILITY

The present disclosure relates to a method of identifying a target gene for tumor therapy by analyzing intratumor heterogeneity, and may be applied to medical fields using a genetic test, etc.

1. A method of identifying a target gene for tumor therapy, the method comprising:
collecting multiple samples from a patient's tumor;
analyzing genetic variations of the multiple samples;
measuring drug sensitivity of each sample by subjecting the multiple samples to drug screening;
analyzing intratumor heterogeneity of the tumor on the basis of the result of analyzing the genetic variations and the result of measuring drug sensitivity; and
identifying the target gene of the tumor on the basis of the result of analyzing the intratumor heterogeneity.

2. The method of identifying the target gene for tumor therapy of claim 1, wherein
the collecting of the multiple samples is collecting of samples from different sites of the patient's tumor.

3. The method of identifying the target gene for tumor therapy of claim 1, wherein
the collecting of the multiple samples is collecting of each sample from the patient's tumor at different times of development.

4. The method of identifying the target gene for tumor therapy of claim 1, wherein
the analyzing of genetic variations of the multiple samples is performed by massive sequencing analysis (next-generation sequencing, NGS).

5. The method of identifying the target gene for tumor therapy of claim 1, wherein
a drug used in the measuring of drug sensitivity is an anticancer agent.

6. The method of identifying the target gene for tumor therapy of claim 1, wherein
the measuring of drug sensitivity of each sample by subjecting the multiple samples to drug screening comprises
obtaining a cell viability curve of each sample according to a dose of each drug; and
calculating an area under the curve.

7. The method of identifying the target gene for tumor therapy of claim 1, wherein
the identifying of the target gene of the tumor comprises
measuring a variance and a mean value of the drug sensitivity for each sample; and
selecting a drug with the highest mean value of the drug sensitivity, among drugs having a variance lower than a predetermined value.

8. The method of identifying the target gene for tumor therapy of claim 1, wherein
the analyzing of intratumor heterogeneity comprises
analyzing intratumor heterogeneity on the basis of the result of analyzing the genetic variations; and verifying the intratumor heterogeneity on the basis of the result of measuring the drug sensitivity.
